(19) United States
(12) Patent Application Publication
(10) Pub. No.: US 2024/0260872 A1
(43) Pub. Date: Aug. 8, 2024

(54) SYSTEM AND METHOD FOR QUANTIFYING A MENTAL STATE

(71) Applicant: Harman International Industries, Incorporated, Stamford, CT (US)

(72) Inventors: Andrey Viktorovich FILIMONOV, Bogorodskiy rayon, Kamenki (RU); Ivan Sergeevich SHISHALOV, Nizhniy Novgorod (RU); Valeriya Alekseevna DEMAREVA, Nizhniy Novgorod (RU); Anastasiya Vladimirovna BAKHCHINA, Nizhniy Novgorod (RU); Anastasiya Sergeevna FILATOVA, Balakhna (RU); Evgeny Pavlovich BURASHNIKOV, Nizhny Novgorod (RU); Anzhela Grigorevna BUROVA, Nizhny Novgorod (RU)

(21) Appl. No.: 18/565,061

(22) PCT Filed: May 28, 2021
(86) PCT No.: PCT/RU2021/000224

§ 371 Date: Nov. 28, 2023

(51) Int. Cl. A61B 5/18; A61B 5/00; A61B 5/16; G16H 50/30

(52) U.S. Cl. CPC A61B 5/18; A61B 5/165; A61B 5/7203; A61B 5/7267; G16H 50/30

(57) ABSTRACT

A computer-implemented method for quantifying a mental state, the method comprising: collecting, as a first training data subset, at least one pair of biosignals, wherein each biosignal is related to an intensity of a mental state of one or more persons; receiving, as a second training data subset, at least one annotation indicative of which biosignal of the pair of biosignals is related to a higher intensity of the mental state; training the artificial neural network on a training dataset comprising the first and second training data subsets to predict values of intensities of mental states; receiving a production dataset input comprising at least one production biosignal related to an intensity of the mental state as an input dataset; and processing the production input dataset by the artificial neural network to predict a value of an intensity of the mental state related to the production biosignal.

### FIELD

The present disclosure relates to quantifying mental states. The methods and systems are usable in health monitoring devices, in particular vehicle-based health monitoring devices.

### BACKGROUND

Determining a mental state of an individual, such as a cognitive load of a driver of a vehicle, is a task relevant in fields including vehicle safety. Therefore, there is an interest for a reliable quantification of a mental state.

The following documents relate to the determination of cognitive load and applications thereof:
US National Highway Traffic Safety Administration report DOT HS 810 635. November 2006.
Euro NCAP, Assessment Protocol—Safety Assist, Version 9.0.1, February 2019
U.S. Pat. No. 9,934,425B2
US20180125405A1
US20180125356A1
U.S. Pat. No. 7,344,251B2
U.S. Pat. No. 6,090,051A
US20180125406A1
U.S. Pat. No. 7,435,227B2
U.S. Pat. No. 7,938,785B2
WO2006024129A1
U.S. Pat. No. 9,723,992B2
U.S. Pat. No. 9,646,046B2
U.S. Ser. No. 10/111,611B2
U.S. Pat. No. 9,763,573B2
WO2015116832A1
U.S. Pat. No. 7,438,418B2
US20070066916A1
WO2008107832A1
U.S. Pat. No. 6,102,870A
US20080150734A1
U.S. Pat. No. 9,642,536B2
U.S. Ser. No. 10/667,723B2
U.S. Pat. No. 8,977,347B2

### SUMMARY

Disclosed and claimed herein are systems and methods for quantifying a mental state.

A first aspect of the present disclosure relates to a computer-implemented method for quantifying a mental state. The method comprises the following steps:

collecting, as a first training data subset, at least one pair of biosignals, wherein each biosignal is related to an intensity of a mental state of one or more persons;
receiving, as a second training data subset, at least one annotation indicative of which biosignal of the pair of biosignals is related to a higher intensity of the mental state;
supplying the first training data subset and the second training data subset to an artificial neural network as a training dataset;
training the artificial neural network on the training dataset to predict values of intensities of mental states;
receiving a production input dataset comprising at least one production biosignal related to an intensity of the mental state as an input dataset; and
processing the production input dataset by the artificial neural network to predict a value of an intensity of the mental state related to the production biosignal.

A mental state refers to a state of mind of one or more persons and may comprise a mood. A mental state may vary gradually in intensity, and the two biosignals indicate two different intensities of the mental state. For example, a mental state may comprise a cognitive load, and the first biosignal may be measured for a person experiencing high cognitive load such as when executing a cognitively demanding task, and the second biosignal may be measured for the same person at a low cognitive load, e.g. when relaxed. However, also other mental states may be chosen, such as stress. Also, mental states are not necessarily limited to one person. Rather, a mental state observed in two persons may be quantified. In that exemplary case, the first biosignal relates one person and the second biosignal relates to the second person. Biosignals may comprise different types of measurable physiological values, such as a heart interbeat interval, or eye openness. Biosignals may be treated immediately after measurement, or pre-recorded and used as an input later.

Annotations comprise binary values, such as Boolean values, that indicate which biosignal is related to a higher intensity of the mental state. For example, the annotation may comprise an indication that the first biosignal is related to a higher intensity of the mental state when the first biosignal is recorded for a person solving a cognitively demanding task, and the second biosignal is recorded for a person not solving a cognitively demanding task.

In a training phase, biosignals and annotations are collected and the artificial neural network is trained to predict values of the annotations. Training may thus comprise predicting, by the artificial neural network, a value for the intensity of the mental state for each biosignal, and adjusting the weights of the artificial neural network such that the predicted intensity for one of the biosignals is higher if the annotation indicates that the biosignal is related to a higher intensity of the mental state. The value for each mental state may preferably be a numerical value. Thereby, by receiving only binary inputs, but training on a dataset comprising a plurality of pairs of biosignals, a qualitative determination of the intensity of a mental state by the artificial neural network is possible.

In an inference phase, a single production biosignal may be treated the intensity of a mental state. Thereby, an artificial neural network trained to predict a value of an intensity of, e.g., cognitive load, may be used to predict cognitive load from new input data.

An advantage of the method is thus that the artificial neural network can be trained on a reliable dataset, since a comparison between two different levels of cognitive load can be made quite reliably. For example, a person can state much more easily, and at a higher level of confidence, which mental state out of two mental states is more intense, as opposed to, e.g., rating a mental state on a scale from one to ten. By training the artificial neural network using such a training dataset, mental states can be predicted reliably.

In an embodiment, the method further comprises pre-processing one or more of the biosignals before supplying the biosignals to the artificial neural network for training and/or processing. Pre-processing comprises removing noise and/or extracting a feature according to one or more predefined criteria.

By pre-processing, steps may be undertaken to reduce the entropy in the data. For example, if a heart interbeat interval is to be determined, camera images of a face of the person may be taken, colour changes over time in one or more positions on the face may be determined, and the signal may be analysed to identify heart interbeat interval by appropriate calculations as known in the art, such as Fourier transform, fitting a model, or using a machine learning based approach to extract the heart interbeat interval. In other embodiments, other types of pre-processing may be used as appropriate for the biosignals.

In a further embodiment, the first training data subset comprises at least two pairs of biosignals comprising the same biosignal. This may imply a step of automatically curating the training dataset. In this embodiment, a first training biosignal is be comprised in two or more different pairs of biosignals in the training dataset, along with annotations indicating the relative intensities. Thereby, the first training biosignal may be compared to two or more different second training signals. This facilitates the training process and reduces the amount of raw data needed for training.

In a further embodiment, training the artificial neural network comprises:
supplying the first training data subset to an input layer of the artificial neural network;
generating, by the artificial neural network, two output values, wherein each output value is indicative of the intensity of a mental state related to one of the biosignals;
comparing the output values to generate a comparator value indicative of which output value is larger; and
training the artificial neural network predict, by the comparator value, the second input training data subset.

The input layer is thus configured to receive two biosignals and process the biosignals. The two output signals quantify intensities of mental states, and comprise preferably numerical values. They are compared to generate a comparator value, e.g. a Boolean value, indicating which value is higher. Training then comprises adjusting the weights such that the comparator value matches the annotation.

In a further embodiment, the artificial neural network comprises two parts. In this embodiment, each part is configured to receive one input biosignal of the pair of biosignals and to generate one output value indicative of the intensity of the mental state related to the input biosignal. Processing the production dataset comprises supplying the production input dataset to at least one of the parts in this embodiment. A part of the artificial neural network may comprise a plurality of nodes to process one of the input signals. The parts may therefore comprise neural networks themselves.

In a further embodiment, the parts are not in communication to each other. During a training phase, the weights are determined separately for each part.

In a further embodiment, the parts comprise identical node structures. Thereby, the artificial neural network is symmetric in structure with respect to the inputs.

In a further embodiment, training comprises determining a common set of weights for the parts. Thereby the artificial neural network is entirely symmetric with respect to swapping the inputs, such that the result is invariant as to whether a production biosignal is sent to the first part or second part. Training may thus lead to faster convergence as the common set of weights is trained for both parts of the artificial neural network.

In a further embodiment, the mental state comprises one or more of stress, readiness, attention, drowsiness, and/or cognitive load. These mental states can all be quantified based on comparisons.

In a further embodiment, the first biosignal and the second biosignal relate to mental states of the same person at different time intervals.

Thereby, an annotation may be generated by one and the same person, who can compare mental states as experienced. For example, a trend may be identified, i.e. the person may state being more stressed at a first time, when the first biosignal is recorded, than at a second time, when the second biosignal is recorded, or vice versa. The annotation may be set accordingly.

In a further embodiment, the first biosignal and the second biosignal relate to mental states of two different persons. Thereby, differences is physiological reaction to a mental state can be determined.

In a further embodiment, training the artificial neural network comprises supervised learning. For example, the weights may be set by backpropagation such that the comparator value predicts the annotation.

In a further embodiment, training the artificial neural network comprises minimising a mean squared error of the comparator value with respect to the second training data subset.

In a further embodiment, the steps of
a. capturing a production input dataset; and/or
b. processing the production input dataset
   are executed by a computer attached to and/or comprised in a mobile device.

In particular, the mobile device may be comprised in a vehicle. Upon inference, the mobile device may thus determine the cognitive load of a driver of a vehicle.

A second aspect of the disclosure relates to a system for quantifying an intensity of a mental state. The system comprises:
a. one or more sensors configured to determine a biosignal;
b. an input device configured to receive an annotation;
c. a processing unit; and
d. a memory comprising instructions that, when executed by the processing unit, cause the system to execute a method of any of the preceding claims.

All properties and embodiments that apply to the first aspect also apply to the second aspect.

### BRIEF DESCRIPTION OF THE DRAWINGS

The features, objects, and advantages of the present disclosure will become more apparent from the detailed description set forth below when taken in conjunction with the drawings in which like reference numerals refer to similar elements.

FIG. 1 shows a flow chart of a method for training an artificial neural network according to an embodiment;

FIG. 2 shows a flow chart of a method for using an artificial neural network in an inference phase for quantifying a mental state according to an embodiment;

FIG. 3 shows a block diagram of a system according to an embodiment; and

FIG. 4 shows a block diagram of a system according to an embodiment.

### DETAILED DESCRIPTION OF THE PREFERRED EMBODIMENTS

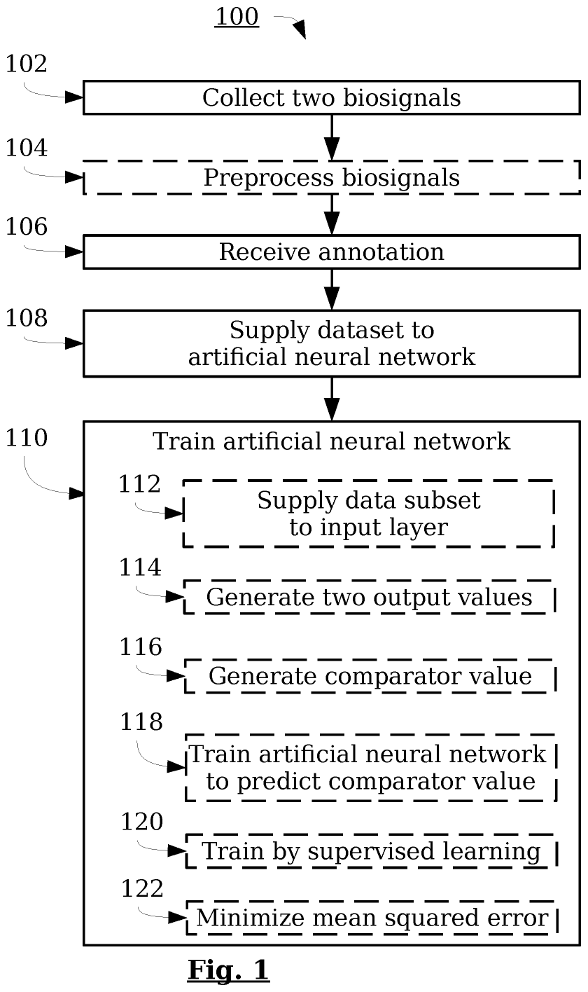

FIG. 1 shows a flow chart of a method 100 for training an artificial neural network according to an embodiment.

The method 100 begins by collecting two biosignals 102. The biosignals carry information on a mental state. For example, a biosignal may comprise a heartbeat signal, which carries some information of a level of stress experienced by an individual. Other examples of biosignals may include an eye blink rate, an eye openness, electrocardiographic data, or other biosignals. Biosignals may be recorded directly from an individual or be pre-recorded and stored in a memory. At least a second biosignal of the mental state is collected. Here, the signals being of the same mental state refers to the signals being of the same category of a mental state, e.g. both biosignals relate to a level of stress so that the biosignals are comparable. The biosignals can, for example, pertain to the same person and be recorded at different times when the person is experiencing the mental state at a different intensity. For example, the person may be a test user subjected to different levels of stress. In an alternative embodiment, the mental state may comprise a cognitive load, which indicates whether the person is solving a demanding cognitive task. In this example, a first biosignal can be recorded when the person is solving a cognitively demanding problem, such as driving a car, or solving a mathematical problem. A second signal may be taken when the person is not solving a problem but in a relaxed state. However, the present disclosure is not restricted to comparing signals from the same person. For example, biosignals of two different persons may be recorded. Then, a difference between the biosignals carries information on their differences in physiological response to a situation. The biosignals may be pre-processed, 104. Pre-processing steps may comprise any kind of pre-processing known in the art. For example, noise may be removed from the data. Features may be extracted by application of a mathematical model. The biosignals form a first training data subset to be supplied to an input layer of the neural network, as detailed below.

At 106, an annotation of a difference in the mental state related to the biosignals is received. The annotation need not comprise quantitative information. However, the annotation comprises a binary value indicating whether the first or the second biosignal relates to a higher intensity of the mental state. If, in an embodiment, the mental state comprises a cognitive load, then the annotation may be a Boolean value that is true if the person was solving a demanding cognitive task when the first biosignal was recorded. The annotation may also be determined by the person in a self-assessment of a current intensity of a mental state. Thereby, advantage is taken from the fact that humans can give more reliable information on whether one out of two mental states is more intense than by quantifying mental states, e.g. on a scale from zero to hundred. The annotations form a second training data subset.

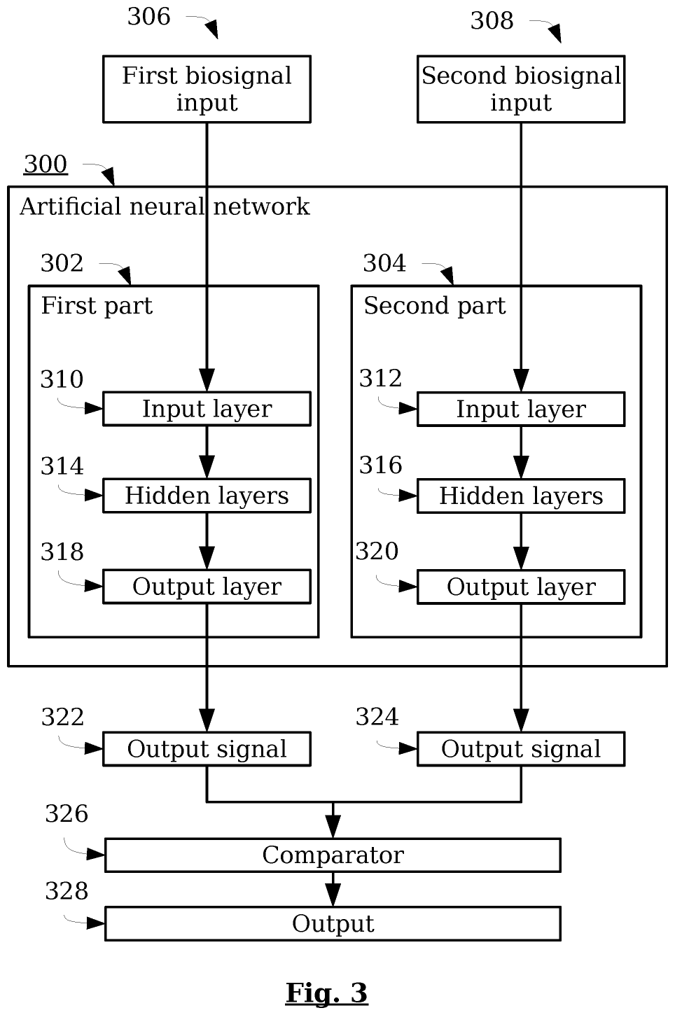

At 108, the training dataset comprising the first training data subset and the second training data subset is supplied to the artificial neural network. The training dataset comprises at least two biosignals and an annotation. However, for training the neural network, preferably a plurality of data triples are used, wherein each triple comprises two biosignals and one annotation indicating which biosignal relates to a higher intensity of the mental state. Using this training dataset, the artificial neural network is trained, 110, to predict a numerical value quantifying the mental state. The output of the neural network therefore comprises at least one numerical value relating to a relative intensity of the mental state, as described with reference to FIG. 3. In an embodiment, a first training data subset comprising the biosignals is supplied to an input layer of the artificial neural network. The input layer is configured to receive two biosignals. By processing two individual biosignals, two output values may be generated, 116, wherein each output value quantifies an intensity of the mental state associated with each of the biosignals. These output signals may then be compared to each other to obtain a comparator value 116, e.g. a Boolean value, which indicates which of the output values is higher. The artificial neural network may then be trained, 118, to yield output values, for which the comparison predicts the annotation received at step 106. Training may comprise techniques of supervised learning, 120. Training may comprise determining and minimising, 122, a mean squared error indicative of a discrepancy between the annotation and the comparator value. However, the present disclosure is not limited to specific types of training. Rather, also other types of training an artificial neural network may be used. By training with sufficiently large training dataset, the weights of the artificial neural network are determined so that the output values contain quantitative information on the intensity of the mental states, although the annotations only comprise binary values on specific pairs of biosignals.

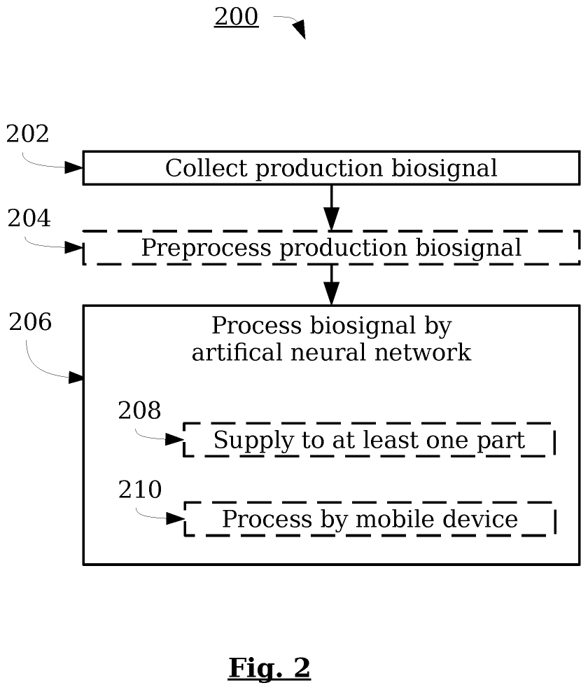

FIG. 2 shows a flow chart of a method 200 for using an artificial neural network in an inference phase for quantifying a mental state according to an embodiment. At 202, a production biosignal is collected. In an embodiment, the production biosignal may comprise a heartbeat signal determined by remote photoplethysmography using camera images of a user. Similarly to step 104, the signal may optionally be pre-processed, 204, which may include determining a heart rate from the camera images. The signal is then processed, 206, to predict a numerical value for the intensity of the mental state as experienced by the user. Processing may be executed by a part of the artificial neural network. The biosignal may thus be supplied, 208, to one part of the artificial neural network, wherein the part comprises an input to receive the production biosignal and an output to yield a determined numerical value for the intensity of the mental state. In an exemplary embodiment, one or more the processing steps may be carried out by a mobile device, 210. However, the processing steps may also be carried out on a stationary computing device, e.g., a desktop or server computer.

FIG. 3 shows a block diagram of a system according to an embodiment. The system comprises an artificial neural network 300. The artificial neural network comprises a first part 302 and a second part 304 in this embodiment. In this embodiment, each part is a distinct neural network and is configured to receive one input signal 306, 308 at an input layer 310, 312, process the input via one or more hidden layers 314, 316 and yield, via an output layer 318, 320, an output signal 322, 324. Thereby, the output signal 322 of the first part 302 indicates an intensity of a mental state related to the first biosignal input 306. Similarly, the output signal 324 of the second part 304 indicates an intensity of a mental state related to the second biosignal input 308. In embodiments, the first part 302 and the second part 304 may comprise identical node structures, e.g. in the hidden layers, so that the first and second parts fulfil equivalent tasks. In an embodiment, the first part and the second part may share a common set of weights, such that upon training each weight is adjusted with respect to two nodes, to increase convergence. Alternatively, the first part 302 and the second part 304 may comprise different weights to reduce the complexity of the training phase.

The output signals 322, 324 quantify the intensity of the associated mental state. They may be expressed as numerical value, or encoded differently. The output signals 322, 324 are supplied to the comparator 326 that yields an output 328 as to which of the output signals 322, 324 is higher. The comparator 326 therefore provides a value 328 that is to predict the annotations of the second training data subset. Training on a training dataset therefore leads to output signals 322 that cause the comparator to yield an accurate prediction of the annotations.

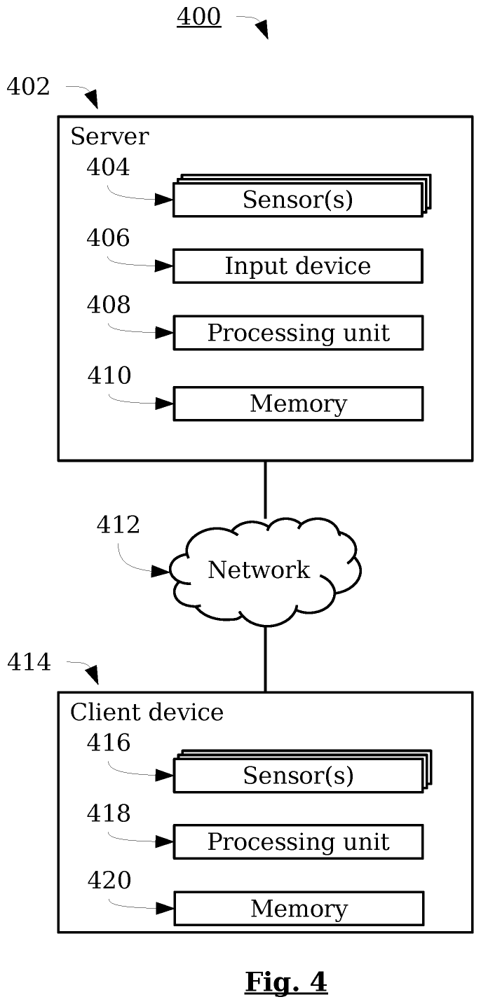

FIG. 4 shows a block diagram of a system 400 according to an embodiment. The system 400 comprises a server configured to execute the training process 100 of FIG. 1 to determine weights for the artificial neural network. The server comprises one or more sensors 404, which may comprise cameras, electrocardiographic devices, or other sensors for biosignals. The server also comprises an input device 406, which is configured to receive an input on which mental state of two mental states in more intense. The processing unit may execute one or more steps of any of the methods discussed above. The memory 410 may comprise instructions to cause the server to execute the steps of any of the methods. The server 402 may be a single computing device or a plurality of computing devices.

The server 402 and the client device 414 are in communication via a network 412. The network enables the client device 414 to exchange data with the server, comprising receiving weights of an updated version of an artificial neural network, and/or sending one or more biosignals to the server for analysis and/or one or more pre-processing steps.

In an embodiment, the client device 414 may be a mobile device. In particular, it may be comprised in a vehicle and configured to determine drowsiness of a driver. The determination of drowsiness may allow the vehicle electronics to react, e.g. by alerting the driver by an acoustic signal or by sending a warning to other vehicles in proximity. In this illustrative example, the sensor may comprise one or more cameras to observe the driver.

In alternative embodiments, the client device 414 may be a stationary device.

From the camera images, an eye openness value may be determined, from which, e.g., a blink rate may be determined as a pre-processing step. Alternatively, a heart rate may be determined by remote photoplethysmography. The biosignal may then either be analysed on the client device by execution of method 200 by the processing unit, or sent via the network to the server 402 for processing.

It should be noted that the present disclosure is not limited to the above embodiment. The methods described with reference to FIGS. 1 and 2 and the artificial neural network of FIG. 3 may be executed on any suitable hardware.

### REFERENCE SIGNS

100 Method for training an artificial neural network
102 Collect two biosignals
104 Pre-process biosignals
106 Receive annotation
108 Supply dataset to artificial neural network
110 Train artificial neural network
112 Supply data subset to input layer
114 Generate two numerical output values
116 Generate comparator value
118 Train to predict second input
120 Train artificial neural network by supervised learning
122 Minimize mean squared error
200 Method for determining a numerical value of a mental state
202 Collect production biosignal
204 Pre-process production biosignal
206 Process production biosignal
208 Supply production input to at least one part
210 Process by mobile device
300 Artificial neural network
302 First part of the artificial neural network
304 Second part of the artificial neural network
310, 312 Input layers
314, 316 Hidden layers
318, 320 Output layers
322, 324 Output signals
326 Comparator
328 Output
400 System
402 Server
404 Sensor(s)
406 Input device
408 Processing unit
410 Memory
412 Network
414 Mobile device
416 Sensor(s)
418 Processing unit
420 Memory

1. Computer-implemented method for quantifying a mental state, the computer-implemented method comprising:
collecting, as a first training data subset, at least one pair of biosignals, wherein each biosignal is related to an intensity of a mental state of one or more persons;
receiving, as a second training data subset, at least one annotation indicative of which biosignal of the at least one pair of biosignals is related to a higher intensity of the mental state;
supplying the first training data subset and the second training data subset to an artificial neural network as a training dataset;
training the artificial neural network on the training dataset to predict values of intensities of mental states;
receiving a production input dataset comprising at least one production biosignal related to an intensity of the mental state as an input dataset; and
processing the production input dataset by the artificial neural network to predict a value of an intensity of the mental state related to the at least one production biosignal.

2. The computer-implemented method claim 1,
further comprising pre-processing one or more of the biosignals before supplying the biosignals to the artificial neural network for training and/or processing, wherein the pre-processing comprises:
removing noise; and/or
extracting a feature according to one or more predefined criteria.

3. The computer-implemented method of claim 1, wherein the first training data subset comprises at least two pairs of biosignals comprising the same biosignal.

4. The computer-implemented method of claim 1, wherein training the artificial neural network comprises:
supplying the first training data subset to an input layer of the artificial neural network;
generating, by the artificial neural network, two output values, wherein each output value is indicative of the intensity of a mental state related to one of the biosignals;
comparing the two output values to generate a comparator value indicative of which output value is larger; and
training the artificial neural network to predict, by the comparator value, the second training data subset.

5. The computer-implemented method of claim 4,
wherein the artificial neural network comprises two parts,
wherein each part is configured to receive one input biosignal of the pair of biosignals and to generate one output value indicative of the intensity of the mental state related to the input biosignal; and
wherein processing the production input dataset comprises supplying the production input dataset to at least one of the two parts.

6. The computer-implemented method of claim 5, wherein the two parts are not in communication to each other.

7. The computer-implemented method of claim 5, wherein the two parts comprise identical node structures.

8. The computer-implemented method of claim 5, wherein training comprises determining a common set of weights for the two parts.

9. The computer-implemented method of claim 1, wherein the mental state comprises:
stress;
readiness;
attention;
drowsiness; and/or
cognitive load.

10. The computer-implemented method of claim 4, wherein a first biosignal and a second biosignal relate to mental states of the same person at different time intervals.

11. The computer-implemented method of claim 4, wherein a first biosignal and a second biosignal relate to mental states of two different persons.

12. The computer-implemented method of claim 1, wherein training the artificial neural network comprises supervised learning.

13. The computer-implemented method of claim 4, wherein training the artificial neural network comprises minimizing a mean squared error of the comparator value with respect to the second training data subset.

14. The computer-implemented method of claim 1, wherein the steps of
receiving a production input dataset; and/or
processing the production input dataset
are executed by a computer attached to and/or comprised in a mobile device.

15. A system for quantifying an intensity of a mental state, the system comprising:
one or more sensors configured to determine a biosignal;
an input device configured to receive an annotation;
a processing unit; and
a memory comprising instructions that, when executed by the processing unit, cause the system to execute a method of claim 1.
